(19) United States
(12) Patent Application Publication
(10) Pub. No.: US 2008/0021319 A1
(43) Pub. Date: Jan. 24, 2008

(54) METHOD OF MODIFYING DATA ACQUISITION PARAMETERS OF AN ULTRASOUND DEVICE

(76) Inventor: James Hamilton, Brighton, MI (US)

Correspondence Address: SCHOX PLC, 209 N. MAIN STREET #200, ANN ARBOR, MI, 48104, US

(21) Appl. No.: 11/781,217

(22) Filed: Jul. 20, 2007

Related U.S. Application Data
(60) Provisional application No. 60/807,876, filed on Jul. 20, 2006.
(60) Provisional application No. 60/807,879, filed on Jul. 20, 2006.
(60) Provisional application No. 60/807,880, filed on Jul. 20, 2006.

(51) Int. Cl. A61B 8/00

(52) U.S. Cl. 600/437

(57) ABSTRACT

In one embodiment, the invention is a method of modifying data acquisition parameters of an ultrasound device and includes the steps of collecting at least two acoustic beams, calculating optimizations for at least one data acquisition parameter using the acoustic beams, modifying data acquisition parameters according to the optimization. In another embodiment, the invention is a method of collecting at least two acoustic beams for an ultrasound device and includes multiplexing multiple transmit beam signals, transmitting the multiplexed transmit beam signals, receiving at least one receive beam corresponding to each transmit beam, and demultiplexing the received beams.

### CROSS-REFERENCE TO RELATED APPLICATIONS

This application claims the benefit of U.S. Provisional Application No. 60,807,876 filed 20 Jul. 2006 and entitled “Multi-Resolution Tissue Tracking”, U.S. Provisional Application No. 60/807,879 filed 20 Jul. 2006 and entitled “Data Acquisition Methods for Ultrasound Based Tissue Tracking”, and U.S. Provisional Application No. 60/807,880 filed 20 Jul. 2006 and entitled “Data Display and Fusion”, where are all incorporated in their entirety by this reference.

### TECHNICAL FIELD

This invention relates generally to the ultrasound field, and more specifically to a new and useful method of data acquisition in the ultrasound field.

### BACKGROUND

Traditional ultrasound acquisition includes a transmit beam from an ultrasound transducer. The beam represents the region insonified by the transmitted ultrasound pulse from the transducer. Characteristics of the pulse and beam are controlled by the beamformer. The receive beam is formed from detected ultrasound echoes created as the transmitted ultrasound pulse propagates. This transmit and receive beam combination may be referred to as an acoustic beam. The beamformer may have dynamic focuses for each range or depth sample. The transmit and receive beams are usually collinear to improve resolution and sensitivity, as shown in FIG. 1.

In contrast, multiple receive beam acquisition collects two or more receive beams for each transmit. The advantage of this method is faster acquisition (i.e., increased frame rate), as larger area can be simultaneously interrogated by ultrasound signals. Multiple receive beam capability is provided by the beamformer, which simultaneously processes multiple receive beams in parallel. In this example, as shown in FIG. 2, four receive beams are produced at the same rate as a single, traditional, transmit/receive beam pair.

Tissue elasticity and strain imaging, including cardiac contractility imaging, may be improved by multiple receive beam acquisition by providing fast acquisition and high image quality for accurate measurement of tissue motion, preferably using speckle tracking. In addition, modification and control of image acquisition characteristics (e.g., transmit and receive beams) may further improve imaging of tissue mechanical properties.

Thus, there is a need in the medical field to modify data acquisition parameters, including increasing acquisition rates, for cardiac contractile imaging and other ultrasound imaging situations. This invention provides such an improved method.

### BRIEF DESCRIPTION OF THE FIGURES

FIG. 1 is a representation of the conventional transmit and receive acquisition.

FIG. 2 is a representation of the conventional multiple receive beam acquisition.

FIG. 3 is a schematic representation of a first preferred embodiment of the invention.

FIG. 4 is a schematic representation of a first version of a second preferred embodiment of the invention.

FIG. 5 is a schematic representation of a second version of a second preferred embodiment of the invention.

FIG. 6 is a representation of the decoding of received signals according to the preferred methods of the invention.

FIG. 7 is a representation of the coded transmit beams according to the preferred methods of the invention.

FIG. 8 is a representation of the frame subset processing according to the preferred methods of the invention.

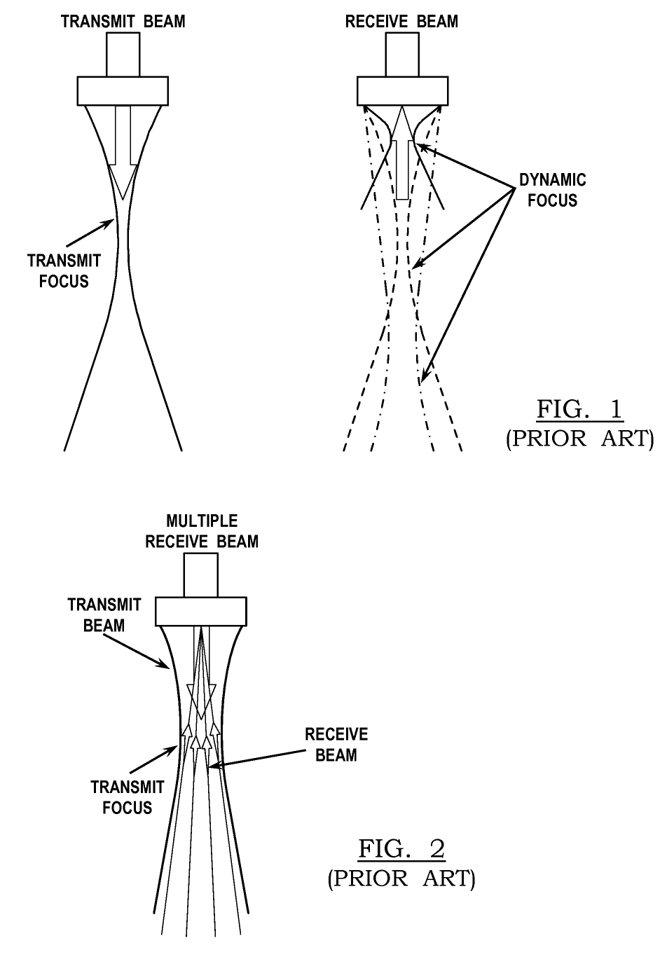

### DESCRIPTION OF THE PREFERRED EMBODIMENTS

The following description of the preferred embodiments of the invention is not intended to limit the invention to these preferred embodiments, but rather to enable any person skilled in the art to make and use this invention.

1. Method of modifying data acquisition parameters

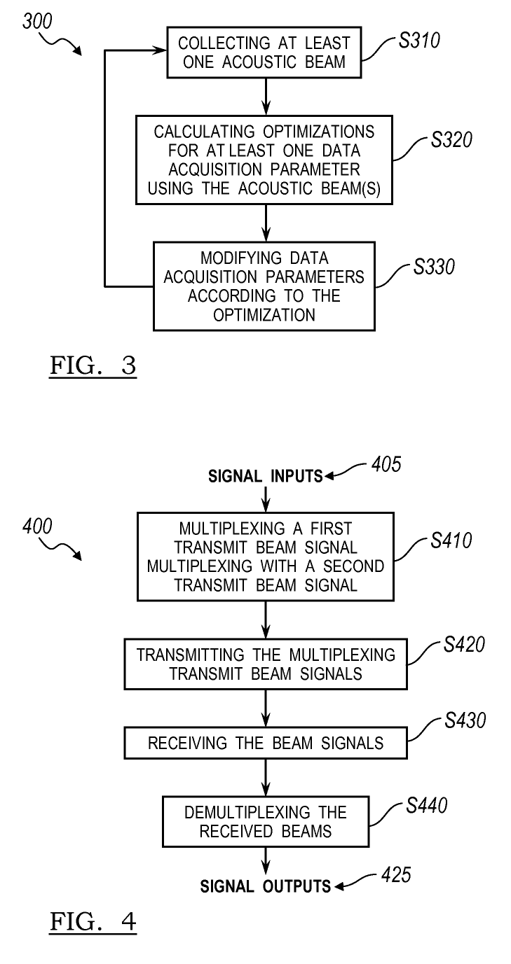

As shown in FIG. 3, the preferred method 300 of modifying data acquisition parameters of an ultrasound device includes collecting at least one acoustic beam S310, calculating optimizations for at least one data acquisition parameter using the acoustic beams S320, and modifying data acquisition parameters according to the optimization(s) S330.

Step S310 functions to collect at least one receive beam. Data collection is controlled by at least one beamformer, which transmits and receives ultrasound signals.

Step S320 functions to calculate optimizations for at least one data acquisition parameter and preferably includes calculating displacement estimates of tissue displacement using speckle tracking. The resulting displacement estimates are preferably used to calculate potential changes to the data collection parameters. For example, previous tracking results may indicate little or no motion in the image or a portion of it. The frame rate or local frame rate may be reduced to lower data rates or trade off acquisition rates with other regions of the image. As another example, the beam spacing can be automatically adjusted to match tissue displacements, potentially improving data quality (i.e., peak correlation). The following data may be used to assess data acquisition parameters: tissue displacement, temporal and spatial variation (e.g., derivatives and variance) of tissue displacement, correlation magnitude, and spatial and temporal variation of correlation magnitude. The following data acquisition parameters may be controlled: transmit and receive beam location, transmit beam width, transmit waveform, and transmit rate (e.g., frame rate). In addition, tissue tracking processing may also be modified based data analysis: beam interpolation, search size, kernel size, and temporal sampling (e.g., processing frame rate).

Step S320 may also include a display of data quality metrics (DQM) to aid the user in optimizing data acquisition. The data quality metric(s) may be presented as a color encoding of the current displayed image mode (e.g., B-mode image). Dual images may be displayed to the user, one current image mode, the other the DQM(s). A global DQM metric maybe calculated and indicated to user by time plot or similar indicator. Data quality metrics are preferably calculated for each sample or sub-set of samples of image region, forming DQM map. The components of the DQM may include: peak correlation, temporal and spatial variation (e.g., derivatives and variance) of tissue displacement, and spatial and temporal variation of correlation magnitude. Operational DQM may be individual or combination of DQM component candidates.

Step S330 functions to modify data acquisition parameters according to the optimization. This is preferably done by communicating changes in the data acquisition parameters to the ultrasound beamformer for implementation. In addition, the user may invoke changes to the acquisition manually based on displayed information such as the DQM. After the adjustments have been made, then Step S310 is preferably repeated; such that new transmit beams are collected using the updated parameters.

2. Method of collecting at least two acoustic beams for high rate acquisition

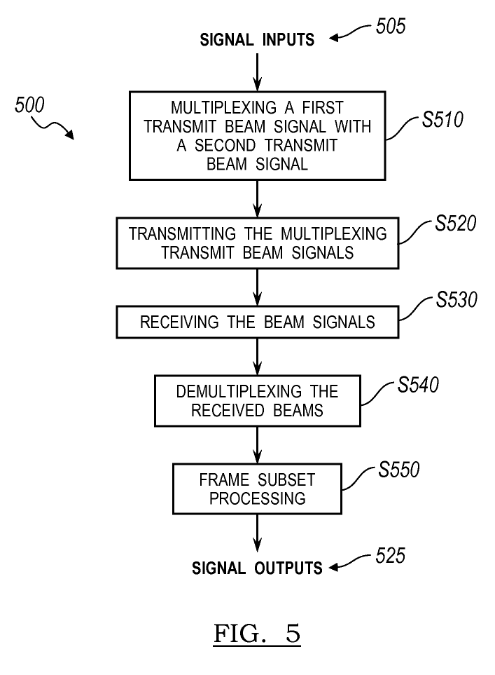

As shown in FIGS. 4-5, the preferred method 400 of collecting at least two acoustic beams include the steps of multiplexing a first transmit beam signal 405 multiplexed with a second transmit beam signal S410, transmitting the multiplexed transmit beam signals S420, receiving at least one receive beam corresponding to the first transmit beam and at least one receive beam corresponding to the second transmit beam signal S430, and demultiplexing the received beams S440 to their respective signals 425. The general method is shown in FIG. 4, while an alternative method is shown in FIG. 5, including the additional step of frame subset processing. The preferred methods of the second embodiment are preferably used to acquire larger frames at faster rates, but may alternatively be used for any suitable purpose.

Step S410 functions to multiplex the transmit beams, preferably to allow multiple transmit beams to be transmitted simultaneously. Preferably the transmit beam signals 405 are modulated with orthogonal or nearly orthogonal codes. More preferably, the transmit beam signals 405 are modulated with pulse codes. The transmit beam signals 405 may, however, be multiplexed with any suitable modulation technique. Preferably the pulse of each transmit beam is encoded to uniquely identify it. In this case two transmit beams are created simultaneously using encoded transmit pulses A and B.

Step S420 functions to transmit the multiplexed beam transmit signals in the ultrasound system.

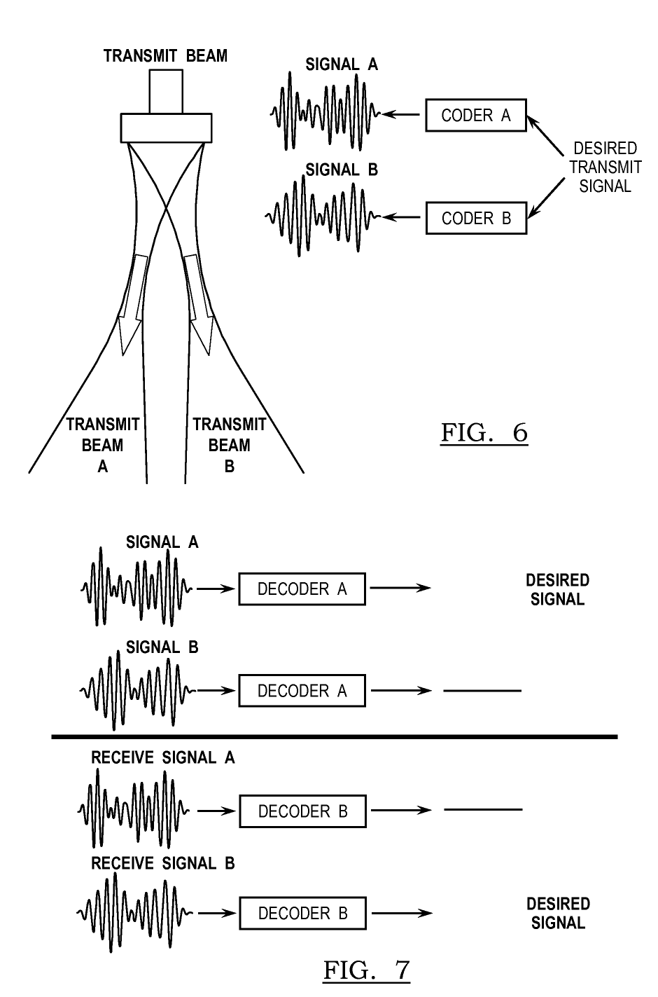

Step S430 functions to detect ultrasound echoes created as the transmitted ultrasound pulse of the multiplexed transmit beam propagates. As shown in FIG. 6, these techniques of the preferred embodiment of the invention increase the data acquisition rate for ultrasound-based tissue tracking by collecting signals in multiple regions simultaneously. During signal reception, all receive beams are preferably collected simultaneously. Alternatively, the receive beams may be collected sequentially.

Step S440 functions to demultiplex the received beams. The processing of signals from multiple receive beams is preferably done in parallel, using coding schemes. The received beam signals are preferably demultiplexed, decoded, demodulated, filtered or “sorted out” into their respective signals 425 using filters specific to the transmit codes. The decoding filters preferably act only on their respective signals 425, rejecting others. As shown in FIG. 7, the original desired transmit signal is returned for decoding filter A operating on code A. No signal is passed for filter A operating on signal B. The same is true for decoding filter B. For good image quality, the codes are preferably nearly orthogonal.

As shown in FIG. 5, the preferred method 500 of collecting at least two acoustic beams may also include frame subset processing S550. Signals 505 and 525, and Steps S510, S520, S530 and S540 of method 500 are preferably identical to Signals 405 and 425, and Steps S410, S420, S440 and S440 of method 400, respectively.

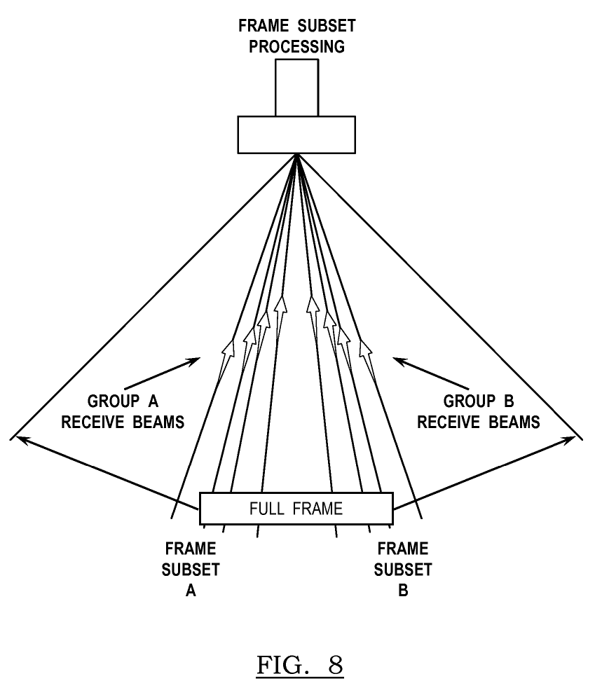

Step S550 functions to process the frame subsets. As shown in FIG. 8, another method to achieve high frame rates needed for accurate ultrasound based tissue (speckle) tracking is to collect subsets of the full frame at a high rate. The local tracking results are then combined to form full frame images at a lower rate. Two regions, A & B, of the full frame are acquired. Beam groups A & B are used to collect these frame subsets. Each group of beams is collected at rates needed for accurate tissue tracking. Other regions of the image are collected in a similar fashion. These techniques are sometimes used for colorflow imaging of blood, which also requires high local frame rates to measure high velocity blood flow. Depending on acquisition time for each beam (e.g., image depth), number of beams in a group and local frame rate, beams from multiple groups maybe collected sequentially. For example the collection scheme could be: beam one from group 1, beam 1 from group 2, beam 2 from group 1, beam 2 from group 2, and so on.

As a person skilled in the art will recognize from the previous detailed description and from the figures and claims, modifications and changes can be made to the preferred embodiments of the invention without departing from the scope of this invention defined in the following claims.

I claim:

1. A method of modifying data acquisition parameters of an ultrasound device, comprising the steps of:
collecting at least one acoustic beam;
calculating optimizations for at least one data acquisition parameter using the acoustic beams; and
modifying data acquisition parameters according to the optimization.

2. The method of claim 1, wherein the step of calculating optimizations for at least one data acquisition parameter includes calculating at least one displacement estimate from the collected acoustic beams using speckle tracking.

3. The method of claim 2, wherein the optimization for at least one data acquisition parameter is at least one data acquisition parameter selected from the group consisting of: beam spacing, local frame rate, full frame rate, transmit and receive beam location, transmit beam width, transmit waveform, transmit rate, collection geometry, and temporal sampling.

4. The method of claim 2, wherein the optimization for at least one data acquisition parameter is beam spacing, and wherein the beam spacing is automatically adjusted based on calculated tissue displacements.

5. The method of claim 2, wherein the step of calculating optimizations for at least one data acquisition parameter using the acoustic beams includes at least one speckle tracking parameter.

6. The method of claim 2 wherein the step of calculating optimizations for at least one data acquisition parameter using the acoustic beams includes at least one speckle tracking parameter, wherein at least one speckle tracking parameter is selected from the group consisting of: beam interpolation, search size, and kernel size.

7. The method of claim 1, wherein the step of calculating optimizations for at least one data acquisition parameter using the acoustic beams also includes calculating an assessment of at least one data acquisition parameter using at least one data type selected from the group consisting of: tissue displacement, temporal and spatial variation of tissue displacement, correlation magnitude, and spatial and temporal variation of correlation magnitude.

8. The method of claim 7, wherein the calculated assessment of at least one data acquisition parameter, using at least one data type, is displayed as at least one image type selected from the group consisting of: color encoding of the current displayed image mode, current image mode, data quality metric image, calculated assessment over time.

9. The method of claim 1, wherein the step of collecting at least two acoustic beams further comprises the steps of:
(a) multiplexing a first transmit beam signal with a second transmit beam signal;
(b) transmitting the multiplexed transmit beam signals;
(c) receiving at least one receive beam corresponding to the first transmit beam signal and at least one receive beam corresponding to the second transmit beam signal; and
(d) demultiplexing the received beams.

10. The method of claim 9 wherein step (a) further includes modulating the transmit beam signals.

11. The method of claim 10 wherein step (a) further includes modulating the transmit beam signals with orthogonal codes.

12. The method of claim 9, wherein step (d) includes demultiplexing the signals by a method selected from the group consisting of: demodulating, masking, filtering, and sorting.

13. The method of claim 9 wherein step (b) includes transmitting the multiplexed beam transmit signals to measure at least one subset of a full ultrasound frame.

14. The method of claim 13, further including the step of combining at least two subsets of a full ultrasound frame into one full frame.

15. A method of collecting at least two acoustic beams for an ultrasound device, comprises the steps of:
(a) multiplexing a first transmit beam signal with a second transmit beam signal;
(b) transmitting the multiplexed transmit beam signals;
(c) receiving at least one receive beam corresponding to the first transmit beam signal and at least one receive beam corresponding to the second transmit beam signal; and
(d) demultiplexing the received beams.

16. The method of claim 15 wherein step (a) further includes modulating the transmit beam signals.

17. The method of claim 16 wherein step (a) further includes modulating the transmit beam signals with orthogonal codes.

18. The method of claim 15, wherein step (d) includes demultiplexing the signals by a method selected from the group consisting of: demodulating, masking, filtering, and sorting.

19. The method of claim 15 wherein step (b) includes transmitting the multiplexed beam transmit signals to measure at least one subset of a full ultrasound frame.

20. The method of claim 19, further including the step of combining at least two subsets of a full ultrasound frame into one full frame.
